(19) United States
(12) Patent Application Publication
(10) Pub. No.: US 2003/0118622 A1
(43) Pub. Date: Jun. 26, 2003

(54) Make-up composition comprising a liquid crystal polymer

(76) Inventor: Roland Ramin, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/267,683

(22) Filed: Oct. 10, 2002

(30) Foreign Application Priority Data
Oct. 10, 2001 (FR) 01 13034

(51) Int. Cl. A61K 7/06; A61K 7/11

(52) U.S. Cl. 424/401; 424/70.16

(57) ABSTRACT

A make up composition comprising, in a cosmetically acceptable medium, particles of a liquid crystal polymer which can be obtained by polymerization of a mixture of monomers comprising at least one first monomer A and at least one chiral second monomer B. A cosmetic process for making up keratinous substances.

The present invention relates to a cosmetic make-up composition comprising particles of a liquid crystal polymer and a process for making up keratinous substances. The composition according to the invention can be applied to human keratinous substances, such as the nails, skin, eyelashes, eyebrows or hair. For example, the invention relates to a nail varnish.

The make-up composition can be, for example, a nail varnish, a blusher, an eye shadow, a foundation, a make-up product for the lips, a make-up product for the body, a mascara, an eye-liner or a make-up product for the eyebrows. The composition can also be applied to make-up accessories, such as false nails, false eyelashes, or wigs, or adherent discs or patches on the skin or lips (of the beauty spot type).

It is known that make-up compositions comprise colouring materials for conferring the desired colour on the composition.

The colouring materials generally used are organic pigments, such as lakes, or inorganic pigments, such as pearlescence agents.

To obtain novel colour effects, make-up compositions comprising interferential pigments, such as polyorganosiloxanes comprising liquid crystal groups, are also known from the documents U.S. Pat. No. 5,851,277 and EP-A-815 826. Interferential pigments are also disclosed in U.S. Pat. Nos. 5,362,315 and 5,851,604. These interferential pigments can produce a colour in a range of hue between at least two specific colours and varying as a function of the incidence of the light and of the angle of observation. Such pigments are sold, for example, under the name “Helicone®” by Wacker, also known under the CTFA name of Polysilicone-12.

However, it has been found that these silicone-comprising interferential pigments cannot make it possible to obtain a satisfactory glossy make-up. For example, these pigments can result in a decline in the gloss of nail varnish films.

The present invention therefore relates to a make-up composition, such as a nail varnish, which can make it possible to obtain a glossy make-up exhibiting an interferential colour effect.

The inventor has found that it is possible to obtain a make-up exhibiting a good gloss property by using particles of a specific liquid crystal polymer.

One aspect of the present invention is a cosmetic composition, such as a make-up composition, comprising, in a cosmetically acceptable medium, particles of a liquid crystal polymer as defined below.

Another aspect of the present invention is a cosmetic process for making up a keratinous substance, such as nails, comprising applying to the keratinous substance the composition as defined above.

Furthermore, another aspect of the present invention is the inclusion of particles of a liquid crystal polymer as defined below in, or for the manufacture of, a cosmetic make-up composition for obtaining a glossy make-up deposited on keratinous substances, such as nails.

Another aspect of the present invention relates to a cosmetic process for making up a keratinous substance comprising applying to the keratinous substance a first coat, also known as base coat, of a first cosmetic composition comprising, in a cosmetically acceptable medium, at least one colouring material and then applying to at least one portion of the said first coat a second coat, also known as top coat, of a second cosmetic composition comprising, in a cosmetically acceptable medium, particles of liquid crystal polymer as defined below, wherein the first composition does not comprise particles of liquid crystal polymer like those present in the second composition.

Another aspect of the present invention relates to a make-up kit comprising:

a first cosmetic composition (also known as “base composition”) comprising, in a cosmetically acceptable medium, at least one colouring material, and

a second cosmetic composition (also known as “top composition”) comprising, in a cosmetically acceptable medium, particles of liquid crystal polymer as defined below, wherein the first composition does not comprise particles of liquid crystal polymer like those present in the second composition,

the first and second compositions being packaged in separate containers.

Another aspect of the present invention relates to a made-up support comprising a make-up, which can be obtained according to one of the make-up processes defined above and is applied to the said support, wherein the said support is chosen, for example, from false nails, false eyelashes, toupees, wigs, and adherent discs and patches on the skin or lips.

The liquid crystal (“LC”) polymer present in the composition according to the invention is a polymer, which can be obtained by polymerization of a mixture of monomers comprising:

a) at least one first monomer A of formula (I)

Y¹-A¹-M¹-A²-Y²

in which

i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,

ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and

iii) M¹ is a group of general formula (I′)

—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, such as 1,4-cyclohexylene groups, and arylene groups comprising from 6 to 10 atoms, such as 1,4-phenylene groups, which are optionally substituted by at least one group chosen from said —B¹, —B² and —B³ groups and heteroarylene groups comprising an aryl nucleus comprising from 6 to 10 atoms which are optionally substituted by at least one group chosen from —B¹, —B² and —B³ groups, the said heteroarylene groups comprising from 1 to 3 heteroatoms chosen from O, N and S atoms, and

b) at least one chiral second monomer B of formula (II) V¹-A′¹-W¹-Z-W²-A′²-V², in which

i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),

and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,

ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and

iii) W¹ and W², which may be identical or different, are each a divalent group chosen from groups of general formula —R′¹—X′¹—R′²—X′²R′³, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, such as 1,4-cyclohexylene groups, and arylene groups comprising from 6 to 10 atoms, such as 1,4-phenylene groups, which are optionally substituted by at least one group chosen from said —B¹, —B² and —B³ groups and heteroarylene groups comprising an aryl nucleus comprising from 6 to 10 atoms which are optionally substituted by at least one group chosen from —B¹, —B² and —B³ groups, the said heteroarylene groups comprising from 1 to 3 heteroatoms chosen from O, N and S atoms,

and Z is a chiral divalent group comprising at least 4 carbon atoms, such as from 4 to 20 carbon atoms and further such as from 4 to 10 carbon atoms (the chiral divalent group comprising at least one asymmetric carbon, such as one or two asymmetric carbons and further such as two asymmetric carbons), and, for example, a chiral divalent group resulting from a group chosen from dihydrohexites, hexoses, pentoses, binaphthyl derivatives (binaphthyl groups), biphenyl derivatives (biphenyl groups), tartaric acid derivatives and glycols which are optically active.

The liquid crystal (“LC”) polymer present in the composition according to the invention can be, for example, a polymer which comprises:

a) at least one residue of a first monomer A of formula (I)

Y¹-A¹-M¹-A²-Y²

in which

i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,

ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and

iii) M¹ is a group of general formula (I′)

—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, such as 1,4-cyclohexylene groups, and arylene groups comprising from 6 to 10 atoms, such as 1,4-phenylene groups, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and

b) at least one residue of a chiral second monomer B of formula (II) V¹-A′¹-W¹-Z-W²-A′²-V², in which

i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),

and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,

ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and

iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, such as 1,4-cyclohexylene groups, and arylene groups comprising from 6 to 10 atoms, such as 1,4-phenylene groups, which are optionally substituted by at least one group chosen from said —B¹, —B² and —B³ groups and heteroarylene groups comprising an aryl nucleus comprising from 6 to 10 atoms which are optionally substituted by at least one group chosen from —B¹, —B² and —B³ groups, the said heteroarylene groups comprising from 1 to 3 heteroatoms chosen from O, N and S atoms,

and Z is a chiral divalent group comprising at least 4 carbon atoms, such as from 4 to 20 carbon atoms and further such as from 4 to 10 carbon atoms (the chiral divalent group comprising at least one asymmetric carbon, such as one or two asymmetric carbons and further such as two asymmetric carbons), and, for example, a chiral divalent group resulting from a group chosen from dihydrohexites, hexoses, pentoses, binaphthyl derivatives (binaphthyl groups), biphenyl derivatives (biphenyl groups), tartaric acid derivatives and glycols which are optically active.

In one embodiment of the present invention, the liquid crystal polymer is obtained by polymerization of a mixture of monomers comprising:

a) at least one first monomer A of formula (I)

Y¹A¹-M¹-A²-Y²

in which

i) Y¹ and Y², which may be identical or different, are each chosen from acrylate and methacrylate groups;

ii) A¹ and A², which may be identical or different, are each a group of formula —CₙH₂ₙ—, wherein n is an integer ranging from 1 to 20, such as ranging from 2 to 6 and further such as an integer equal to 4;

iii) M¹ is a group of general formula (I′)

—R¹—X¹—R²—X²—R³—X³—R⁴—, wherein R¹ and R⁴ are each —O—, R² and R³ are each a —COO— group and X¹, X² and X³ are each a 1,4-phenylene group, wherein the carbonyl group —CO— of R² and of R³ are bonded to the X¹ group and to the X³ group respectively, and

b) at least one chiral second monomer B of formula (II) V¹-W¹-Z-W²-V², in which

i) V¹ is a group chosen from acrylate and methacrylate groups, such as acrylate groups, and V² is a group chosen from C₁-C₂₀ alkyl groups, C₁-C₂₀ alkoxy, (C₁-C₂₀)alkoxycarbonyl and —OH groups. For example, V² is chosen from C₁-C₂₀ alkoxy groups, such as C₁-C₄ alkoxy groups, and further such as a methoxy group;

ii) W¹ is a divalent group of formula —X′¹—CO—O—,

W² is a divalent group of formula —O—CO—X′¹—,

wherein X′¹ is a 1,4-phenylene group,

and Z is a chiral group comprising two bonds, resulting from the dianhydrohexite group, such as a divalent radical of formula:

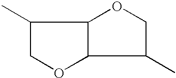

In the definitions of the first monomer A described above, it is understood that the possibility of having two or more oxygen atoms bonded together are excluded.

In one embodiment of the present invention, the mixture of monomers comprises the first monomer A in a concentration ranging, for example, from about 70 to about 99% by weight and the second monomer B in a concentration ranging, for example, from about 1 to about 30% by weight with respect to the total weight of the first monomer A and the second monomer B. And further in another embodiment of the present invention, the mixture of monomers comprises the first monomer A in a concentration ranging, for example, from about 90 to about 95% by weight and the second monomer B in a concentration ranging, for example, from about 5 to about 10% by weight with respect to the total weight of the first monomer A and the second monomer B.

The concentration of the polymerizable groups present in the mixture of the first monomer A and the second monomer B (polymerizable groups Y¹ and Y² of the first monomer A and polymerizable groups V¹ and V² of the second monomer B) ranges, for example, from 3.2 to 15 mmol/g.

According to one embodiment of the present invention, the liquid crystal polymer is obtained from a mixture of the first monomer A and of the second monomer B compriseing polymerizable groups, at least 90% of which are present in monomers comprising at least two polymerizable groups, in a concentration ranging, for example, from 3.2 to 15 mmol/g.

In another embodiment of the present invention, the liquid crystal polymer essentially consists of or consists of a mixture of residues of the monomers A and B that are defined above.

In another embodiment of the present invention, the liquid crystal polymer can exhibit a helical pitch of greater than 450 nm, for example, ranging from about 455 nm to about 5 000 nm, further, for example, ranging from about 455 nm to about 1 000 nm and even further, for example, ranging from about 455 nm to about 650 nm. The helical pitch can readily be determined by one of ordinary skill in the art using known techniques.

The first monomer A may have a weight-average molecular weight ranging, for example, from about 150 to about 800 and further, for example, from about 460 to about 625. In one embodiment of the present invention, the first monomer A is chosen from unsubstituted hydroquinone dibenzoate derivatives.

The second monomer B may have a weight-average molecular weight ranging, for example, from about 500 to around 1 000 and such as, for example, from about 500 to about 700.

The liquid crystal polymer can have, as a further example, a weight-average molecular weight of less than 625.

The liquid crystal polymer defined above may be prepared from the monomer mixture described above according to the processes known in the state of the art, such as those disclosed in U.S. Pat. Nos. 5,362,315 and 5,807,497.

For example, a mixture of the monomers A and B defined above is applied to a smooth base and is oriented; three dimensional crosslinking is carried out by polymerization of the mixture of monomers, and the base is detached.

The mixture of monomers can be applied in a thickness ranging, for example, from about 3 to about 15 μm and further, for example, from 3 to 6 μm to the smooth surface.

The orientation can, for example, be carried out by shearing, using a scraper or a roller.

The polymerization of the oriented mixture of monomers can be carried out in a way already known, for example, in the free radical fashion with the use of commercially available thermal initiators, using electron beams or UV light in combination with commercially available photoinitiators, or by addition or condensation reactions.

The crosslinking of the mixtures of monomers, in the chiral structural state, may, for example, be carried out by means of a polyreaction which, according to the type of the polymerizable, polycondensable or polyadditionable groups, may take place in the form of a radical, ionic or metal-catalyzed polymerization or of a polycondensation reaction or of a polyaddition reaction.

The radical polymerization can be initiated by means of corresponding initiators or by UV radiation, using commercially available photoinitiators, or by high energy radiation, such as electron radiation. An advantage of the thermal polymerization of radicals or of the polymerization via curing with electron beams is that an agent for protecting against light, such as a UV (UVA) absorber or radical scavengers (HALSs), can also be added to the polymerization mixture to stabilize the resulting films or pigments in the face of UV light, for example, for external applications, without resulting in losses as regard to the polymerization conversion, as is the case during curing with UV radiation because of the effect of screening of the photoinitiator by UVA radiation. There can thus be no decrease in the crosslinking density.

If the LC films are cured using peroxide or by electron radiation, the mixture of monomers, for example, further comprises commercially available agents for protecting against light, such as UV absorbers or radical scavengers, in an overall concentration ranging from about 0.5 to about 5% by weight relative to the total weight of the mixture of monomers and the agents.

In addition to the photostabilizers, the mixture of monomers can also comprise other normal additives targeted at inhibiting oxidation or at inhibiting polymerization or additives targeted at improving the rheological properties. Furthermore, absorbent fillers, such as pigments or soot, and also dyes or fluorescence pigments, may be present.

The film obtained after the polymerization is subsequently milled into particles, for example, in the form of platelets.

In one embodiment of the present invention, the particles of liquid crystal polymer have the largest dimension ranging from about 1 μm to about 3 mm and, for example, ranging from about 30 μm to about 500 μm. These particles may be in the form of platelets.

The particles can be separated (sorted) by a process with selectivity for the grain size.

Such polymers and their particles are disclosed in Application EP-A-1 046 692.

Among particles of liquid crystal polymer, mention may be made of those known under the CTFA name Polyacrylate-4 and sold under the names “Helicone® HC Sapphire”, “Helicone® HC Scarabeus”, “Helicone® HC Jade”, “Helicone® HC Maple”, “Helicone® HC XL Sapphire”, “Helicone® HC XL Scarabeus”, “Helicone® HC XL Jade” and “Helicone® HC XL Maple” by Wacker.

The particles of liquid crystal polymer can be present in the composition according to the invention in a concentration, for example, ranging from about 0.01% to about 99% by weight relative to the total weight of the composition, further, for example, ranging from about 0.1% to about 60% by weight, even further, for example, ranging from about 1% to about 30% by weight and even further, for example, ranging from about 5% to about 15% by weight relative to the total weight-of the composition.

The composition according to the present invention, such as the base composition and the top composition, can comprise a cosmetic medium chosen from hydrophilic cosmetic mediums and lipophilic cosmetic mediums.

The composition of the present invention, such as the base composition and the top composition, can comprise water or a mixture of water and of hydrophilic organic solvents, such as alcohols and, for example, alcohols chosen from linear and branched lower monoalcohols comprising from 2 to 5 carbon atoms, such as ethanol, isopropanol and n-propanol, and polyols, such as glycerol, diglycerol, propylene glycol, sorbitol, pentylene glycol and polyethylene glycols. The hydrophilic phase can further comprise hydrophilic C₂ ethers and C₂-C₄ aldehydes. The water or the mixture of water and of hydrophilic organic solvents can be present in the composition according to the present invention, such as the base composition and the top composition, in a concentration ranging from 0% to 90%, such as from about 0.1% to about 90%, by weight relative to the total weight of the composition, and further, for example, ranging from 0% to 60% by weight, such as ranging from about 0.1% to about 60% by weight relative to the total weight of the composition. In one embodiment of the present invention, for example, the composition may comprise a hydrophilic continuous phase.

The composition according to the present invention, such as the base composition and the top composition, can further comprise a fatty phase comprising, for example, fatty substances chosen from fatty substances which are liquid at ambient temperature (generally 25° C.) and fatty substances which are solid at ambient temperature, such as waxes, pasty fatty substances, gums and their mixtures. This fatty phase can further comprise at least one lipophilic organic solvent. The fatty phase can form the continuous phase of the composition according to the invention, such as the base composition and the top composition. The composition can be anhydrous.

Representative fatty substances which are liquid at ambient temperature, often known as oils, and which can be used in the present invention, includehydrocarbonaceous oils of animal origin, such as perhydrosqualene; vegetable hydrocarbonaceous oils, such as liquid triglycerides of fatty acids comprising 4 to 10 carbon atoms, such as triglycerides of heptanoic or octanoic acids, or sunflower, maize, soybean, grape seed, sesame, apricot, macadamia, castor or avocado oils, triglycerides of caprylic/capric acid, jojoba oil or karite butter oil; linear or branched hydrocarbons of mineral or synthetic origin, such as liquid paraffins and their derivatives, liquid petrolatum, polydecenes or hydrogenated polyisobutene, such as parleam; synthetic esters and ethers, for example, of fatty acids, such as purcellin oil, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl maleate, triisocetyl citrate or heptanoates, octanoates or decanoates of fatty alcohols; polyol esters, such as propylene glycol dioctanoate, neopentyl glycol diheptanoate or diethylene glycol diisononanoate; pentaerythritol esters; fatty alcohols comprising from 12 to 26 carbon atoms, such as octyidodecanol, 2-butyloctanol, 2-hexyldecanol, 2-undecylpentadecanol or oleyl alcohol; partially hydrocarbonaceous and/or silicone-comprising fluorinated oils; silicone oils, such as volatile or nonvolatile and linear or cyclic polymethylsiloxanes (PDMS) which are liquid or pasty at ambient temperature, such as cyclomethicones, dimethicones, optionally comprising a phenyl group, such as phenyl trimethicones, phenyltrimethylsiloxydiphenylsiloxanes, diphenylmethyldimethyltrisiloxanes, diphenyl dimethicones, phenyl dimethicones or polymethylphenylsiloxanes; or their mixtures thereof.

These oils may be present in a concentration ranging, for example, from about 0.01 to about 90% and further, for example, from about 0.1 to about 85% by weight with respect to the total weight of the composition.

The composition according to the present invention, such as the base composition and the top composition, can further comprise at least one cosmetically acceptable (acceptable tolerance, acceptable toxicology and acceptable feel) organic solvent. The at least one organic solvent can be present in a concentration ranging, for example, from about 0.1 to about 90%, and further, for example, from about 0.1 to about 60% by weight relative to the total weight of the composition and, even further, for example, ranging from about 0.1 to about 30% by weight relative to the total weight of the composition.

Among organic solvents which can be used in the composition of the present invention, such as the base composition and the top composition, mention may be made of:

ketones which are liquid at ambient temperature, such as methyl ethyl ketone, methyl isobutyl ketone, diisobutyl ketone, isophorone, cyclohexanone or acetone;

alcohols which are liquid at ambient temperature, such as ethanol, isopropanol, diacetone alcohol, 2-butoxyethanol or cyclohexanol;

glycols which are liquid at ambient temperature, such as ethylene glycol, propylene glycol, pentylene glycol or glycerol;

propylene glycol ethers which are liquid at ambient temperature, such as propylene glycol monomethyl ether, propylene glycol monomethyl ether acetate or dipropylene glycol mono(n-butyl) ether;

short-chain esters (comprising from 3 to 8 carbon atoms in total), such as ethyl acetate, methyl acetate, propyl acetate, n-butyl acetate or isopentyl acetate;

ethers which are liquid at ambient temperature, such as diethyl ether, dimethyl ether or dichlorodiethyl ether;

alkanes which are liquid at ambient temperature, such as decane, heptane, dodecane or cyclohexane;

cyclic aromatic compounds which are liquid at ambient temperature, such as toluene and xylene;

aldehydes which are liquid at ambient temperature, such as benzaldehyde or acetaldehyde; and

their mixtures thereof.

These organic solvents can be suitable for making up the nails. As a result, the composition can be used for forming a nail varnish.

The at least one organic solvent may be present in the composition according to the present invention, such as the base composition and the top composition, in a concentration ranging, for example, from about 30% to about 99% by weight relative to the total weight of the composition and further for example, ranging from about 60% to about 90% by weight relative to the total weight of the composition.

The composition of the present invention, such as the base composition and the top composition, can further, for example, comprise a fatty substance which is solid or pasty at ambient temperature, such as gums or waxes.

The waxes can be hydrocarbonaceous, fluorinated and/or silicone-comprising and can be of vegetable, mineral, animal and/or synthetic origin. For example, the waxes may exhibit a melting temperature of greater than 25° C., such as greater than 45° C. Among waxes which can be used in the composition of the invention, mention may be made of beeswax, carnauba wax, candelilla wax, paraffin wax, microcrystalline waxes, ceresin or ozokerite; synthetic waxes, such as polyethylene or Fischer-Tropsch waxes, or silicone waxes, such as alkyl or alkoxy dimethicones comprising from 16 to 45 carbon atoms.

The gums are generally high molecular weight polydimethylsiloxanes (PDMS) or cellulose gums or polysaccharides and the pasty substances are generally hydrocarbonaceous compounds, such as lanolins and their derivatives or PDMSs.

The nature and the amount of the solid substances depend on the desired mechanical properties and the desired textures. By way of illustration, the composition can comprise waxes in a concentration ranging, for example, from about 0.1 to about 50% by weight relative to the total weight of composition and further, for example, from about 1 to about 30% by weight relative to the total weight of composition.

The composition according to the present invention, such as the base composition and the top composition, can further comprise at least one film-forming polymer. The term “film-forming polymer” is understood to mean a polymer capable of forming, by itself alone or in the presence of an additional agent which is able to form a film, a continuous film which adheres to a support, such as to keratinous substances.

The at least one film-forming polymer can be chosen from free radical polymers, polycondensates and polymers of natural origin. The at least one film-forming polymer can be dissolved or dispersed in the form of solid particles in the cosmetically acceptable medium of the composition. For example, the polymer can be provided in the form of solid particles in aqueous dispersion.

The term “free-radical film-forming polymer” is understood to mean a polymer obtained by polymerization of monomers comprising unsaturation, for example, ethylenic unsaturation, each monomer being capable of homopolymerizing (unlike polycondensates).

The film-forming polymers of free-radical type may, for example, be chosen from vinyl polymers and copolymers, such as acrylic polymers.

The vinyl film-forming polymers can also result from the homopolymerization or from the copolymerization of at least one monomer chosen from vinyl esters and styrene monomers.

Among vinyl esters, mention may be made of vinyl acetate, vinyl neodecanoate, vinyl pivalate, vinyl benzoate and vinyl t-butylbenzoate.

Among styrene monomers, mention may be made of styrene and α-methylstyrene.

Among film-forming polycondensates, mention may be made of polyurethanes, polyesters, polyesteramides, short-chain polyesters, polyamides, epoxy ester resins, the resins resulting from the condensation of formaldehyde with an arylsulphonamide, or arylsulphonamide-epoxy resins.

The optionally modified polymers of natural origin may be chosen from shellac resin, sandarac gum, dammars, elemis, copals, cellulose polymers, such as nitrocellulose, cellulose acetate, cellulose acetate/butyrate, cellulose acetate/propionate or ethyl cellulose, and their mixtures.

Among film-forming polymers, mention may be made, for example, of acrylic polymers, polyurethanes, polyesters, polyamides, polyureas or cellulose polymers.

The at least one film-forming polymer can be present in the composition according to the present invention, such as the base composition and the top composition, in a concentration on a dry basis of polymer ranging, for example, from about 0.1% to about 60% by weight relative to the total weight of the composition, and further, for example ranging from about 0.5% to about 40% by weight and even further, for example, ranging from about 1% to about 30% by weight relative to the total weight of the composition.

An additional agent which is able to form a film may be present in the composition to improve the film-forming properties of the composition.

Such an additional agent which is able to form a film can be chosen from any compound known to a person skilled in the art as being capable of fulfilling the desired role and can, for example, be chosen from plasticizers.

In addition, when the composition according to the present invention comprises a film-forming polymer in the form of particles dispersed in the corresponding medium of the composition, the additional agent which is able to form a film can also be chosen from coalescing agents.

The composition according to the present invention, such as the base composition and the top composition, can further comprise at least one additional colouring material different from the particles of liquid crystal polymer which are described above. The at least one colouring material can be chosen, for example, from dyes (water-soluble or fat-soluble) and pulverulent colouring materials, such as pigments, pearlescence agents and glitter, well known to a person skilled in the art. The at least one additional colouring material can be present in the composition according to the present invention, such as the base composition and the top composition, in a concentration ranging, for example, from about 0.01% to about 25% by weight relative to the total weight of the composition. According to one embodiment of the present invention, the base composition can be black in colour.

The term “pigments” should be understood as meaning white or coloured and inorganic or organic particles of any shape which are insoluble in the physiological medium and which are intended to colour the composition.

The term “pearlescence agents” should be understood as meaning iridescent particles of any shape, such as those produced by certain molluscs in their shells or synthesized.

The pigments can be present in the composition according to the present invention, such as the base composition and the top composition, in a concentration ranging from 0 to 15%, for example, from about 0.01% to about 15% by weight relative to the total weight of the composition, and further, for example, from about 0.01% to about 10% by weight and even further, for example, from about 0.02% to about 5% by weight relative to the total weight of the composition.

The pigments can be white or coloured and inorganic or organic. Among inorganic pigments, mention may be made of titanium dioxide, optionally surface treated, zirconium or cerium oxides, zinc, iron or chromium oxides (the iron oxides being black, yellow or red), manganese violet, ultramarine blue, chromium hydrate, ferric blue or metal powders, such as aluminium powder or copper powder.

Mention may be made, among organic pigments, of carbon black, pigments of D & C type, and lakes based on cochineal carmine or barium, strontium, calcium or aluminium.

The pearlescence agents can be present in the composition according to the present invention, such as the base composition and the top composition, in a concentration ranging from 0 to 25%, for example, from about 0.01% to about 25% by weight relative to the total weight of the composition, and further, for example, ranging from about 0.01% to about 15% by weight and even further, for example from about 0.02% to about 5% by weight relative to the total weight of the composition.

The pearlescent pigments can be chosen from white pearlescent pigments, such as mica covered with titanium oxide or mica covered with bismuth oxychloride, coloured pearlescent pigments, such as titanium oxide-coated mica covered with iron oxides, titanium oxide-coated mica covered with, for example, ferric blue or with chromium oxide, or titanium oxide-coated mica covered with an organic pigment of the abovementioned type, and pearlescent pigments based on bismuth oxychloride.

The fat-soluble dyes are, for example, chosen from soybean oil, Sudan brown, DC Yellow 11, DC Orange 5, quinoline yellow, Sudan red III (CTFA name D&C Red 17), lutein, quinizarin green (CTFA name DC Green 6), alizurol purple SS (CTFA name DC Violet No. 2), carotenoid derivatives, such as lycopene, β-carotene, bixin and capsantein, and mixtures thereof.

The composition according to the present invention, for example, the base composition and the top composition, can further comprise fillers. The term “fillers” should be understood as meaning colourless or white and inorganic, organic or synthetic particles of any shape which are insoluble in the medium of the composition, whatever the temperature at which the composition is manufactured. These fillers are used, for example, to modify the rheology or the texture of the composition.

The fillers can be inorganic or organic of any shape, platelet, spherical or oblong, whatever the crystallographic form (for example sheet, cubic, hexagonal, orthorhombic, and the like). Mention may be made of talc, mica, silica, kaolin, polyamide (Nylon®) powders (Orgasol® powders from Atochem), poly(β-alanine) powders, polyethylene powders, tetrafluoroethylene polymer (Teflon®) powders, lauroyllysine, starch, boron nitride, polymeric hollow microspheres, such as those of poly(vinylidene chloride)/acrylonitrile, for example Expancel® (Nobel Industrie), of acrylic acid copolymers (Polytrap® from Dow Corning) and silicone resin microbeads (Tospearls® from Toshiba, for example), particles of polyorganosiloxane elastomers, precipitated calcium carbonate, magnesium carbonate and basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, for example, from 12 to 18 carbon atoms, for example zinc stearate, magnesium stearate, lithium stearate, zinc laurate or magnesium myristate.

The composition according to the present invention, for example, the base composition and the top composition, can be provided, for example, in the form of a suspension, dispersion, solution, gel or emulsion, such as oil-in-water (O/W) or water-in-oil (W/O) emulsion or multiple (W/O/W or polyol/O/W or O/W/O) emulsion, or in the form of a cream, paste, foam, dispersion of vesicles, such as of ionic or nonionic lipids, two-phase or multiphase lotion, spray, powder or paste, such as a soft paste (for example, a paste having a dynamic viscosity at 25° C. of the order of about 0.1 to about 40 Pa.s under a shear rate of 200 s⁻¹, after measuring for 10 minutes in cone/plate geometry). The composition can have an organic continuous phase, for example, an anhydrous phase.

Persons skilled in the art can choose the appropriate dosage form, and its method of preparation, on the basis of their general knowledge, taking into account, on the one hand, the nature of the constituents used, such as their solubility in the support, and, on the other hand, the application envisaged for the composition.

The composition according to the present invention, for example, the base composition and the top composition, can further comprise ingredients commonly used in cosmetics, such as vitamins, thickeners, surfactants, trace elements, moisturizers, softeners, sequestering agents, fragrances, basifying or acidifying agents, preservatives, antioxidants, UV screening agents, or their mixtures.

Of course, a person skilled in the art will take care to choose this or these optional additional compounds, and/or their amount, so that the advantageous properties of the composition according to the present invention cannot, or cannot substantially, detrimentally affected by the envisaged addition.

The composition of the present invention can be obtained according to conventional preparation processes used in cosmetics.

The following examples serve to illustrate, but not to limit, the invention:

### EXAMPLE 1

A nail varnish having the following composition was prepared:

| Nitrocellulose | | 10 | g |
|---|---|---|---|
| Plasticizers and resins | | 15 | g |
| Thickening agent | | 1.5 | g |
| Particles of LC polymer which are sold under the name | | 3 | g |
| “Helicone ® HC Maple” by Wacker | | | |
| Isopropyl alcohol | | 5 | g |
| Ethyl acetate | | 20 | g |
| Butyl acetate | q.s. for | 100 | g |

After application of the composition to the nails, a make-up film was obtained which exhibits a very glossy changeable appearance.

### EXAMPLE 2

A nail varnish having the following composition was prepared:

| Nitrocellulose | | 10 | g |
|---|---|---|---|
| Plasticizers and resins | | 15 | g |
| Thickening agent | | 1.5 | g |
| Particles of LC polymer which are sold under the name | | 10 | g |
| “Helicone ® HC Scarabeus XL” by Wacker | | | |
| Isopropyl alcohol | | 5 | g |
| Ethyl acetate | | 20 | g |
| Butyl acetate | q.s. for | 100 | g |

After application of the composition to the nails, a red-coloured make-up film was obtained which exhibits a very glossy changeable appearance.

### COMPARATIVE EXAMPLES 3 AND 4

A nail varnish according to the present invention (Example 3) and a nail varnish not forming part of the present invention (Example 4) having the following compositions were prepared respectively (the contents are expressed in grams):

| Ingredient | Example 3 | Example 4 |
|---|---|---|
| Nitrocellulose | 11 | 11 |
| Tosylamide/formaldehyde resin | 10 | 10 |
| Tributyl acetylcitrate | 5.7 | 5.7 |
| Bentonite | 1.4 | 1.4 |
| Isopropyl alcohol | 5.5 | 5.5 |
| Red pigment | 0.014 | 0.07 |
| Particles of LC polymer (Helicone ® | 5 | 0 |
| HC Maple XL from Wacker) | | |
| Pearlescent pigment (Mica-titanium | 0 | 0.6 |
| oxide-brown iron oxide) | | |
| Ethyl acetate | 31.5 | 31.5 |
| Butyl acetate | q.s. for 100 | q.s. for 100 |

After application of each varnish to the nails, it was found that the nail varnish of Example 3 according to the present invention makes it possible to obtain a much glossier make-up than that obtained with the varnish of Example 4, which does not comprise the particles of liquid crystal polymer.

### COMPARATIVE EXAMPLES 5 AND 6

A nail varnish according to the present invention (Example 5) and a nail varnish not forming part of the present invention (Example 6) having the following compositions were prepared respectively (the contents are expressed in grams):

| Ingredient | Example 5 | Example 6 |
|---|---|---|
| Nitrocellulose | 10.6 | 10.6 |
| Tosylamide/formaldehyde resin | 8 | 8 |
| Tributyl acetylcitrate | 5.4 | 5.4 |
| Bentonite | 1 | 1 |
| Pyrogenic silica | 1.5 | 1.5 |
| Mica-brown iron oxide (Cloisonné | | |
| Sparkle Rouge 450J from Engelhard) | 2 | 2 |
| Particles of LC polymer (Helicone ® | | |
| HC Jade XL from Wacker) | 1.61 | 0 |
| Interferential pigment Polysilicone-12 | | |
| (Helicone ® 8575 from Wacker) | 0 | 1.61 |
| Isopropyl alcohol | 4.9 | 4.9 |
| Ethyl acetate | 26.9 | 26.9 |
| Butyl acetate | q.s. for 100 | q.s. for 100 |

The gloss of each nail varnish was measured for a wet film with a thickness of 300 μm deposited on the black zone of a contrast card of Leneta Form 1 A-Penopac type and then dried for 24 hours. The measurement was made using a gloss meter of micro-TRI-gloss type from Byk Gardner at a light beam angle of 60°. The following results were obtained:

Example 5: 58.8

Example 6: 44

It was found that the varnish of Example 5 according to the present invention makes it possible to obtain a glossier make-up than that obtained with a varnish of Example 6, not forming part of the present invention.

### EXAMPLE 7

The following two nail varnish compositions A and B were prepared (contents expressed in grams):

| | Composition A | Composition B |
|---|---|---|
| | Base coat | Top coat |
| Nitrocellulose | 10 | 10 |
| Plasticizers and resins | 5 | 5 |
| Thickening agent | 1.5 | 1.5 |
| Black iron oxide | 3 | 0 g |
| Particles of LC polymer (Helicone ® | 0 | 0.5 |
| HC Sapphire from Wacker) | | |
| Particles of LC polymer (Helicone ® | 0 | 1.5 |
| HC Scarabeus from Wacker) | | |
| Isopropyl alcohol | 5 | 5 |
| Ethyl acetate | 20 | 20 |
| Butyl acetate | q.s. for 100 | q.s. for 100 |

A base coat of the composition A was applied to the nail and then, after drying, a top coat of the composition B was applied to the nail. A glossy make-up exhibiting an interferential effect on a black background was obtained.

### EXAMPLE 8

A compact powder having the following composition was prepared:

| Yellow iron oxide | | 1 | g |
|---|---|---|---|
| Brown iron oxide | | 0.55 | g |
| Calcium carbonate hydrate | | 2 | g |
| Polydimethylsiloxane (Dow Corning Fluid 200-10 cSt | | 6.8 | g |
| from Dow Corning) | | | |
| Mixture of trimethylsiloxysilicate resin and of | | 2 | g |
| polydimethylsiloxane (33/67) (Dow Corning 593 Fluid | | | |
| from Dow Corning) | | | |
| Polymethylcetyldimethylsiloxane (Abil Wax 9801 from | | 1.2 | g |
| Goldschmidt) | | | |
| Mica | | 30 | g |
| Particles of LC polymer which are sold under the name | | 5 | g |
| “Helicone ® HC Scarabeus XL” by Wacker | | | |
| Talc | q.s. for | 100 | g |

The pulverulent mixture was compacted in a dish. A compact cosmetic powder was thus obtained, which can be used as eye shadow or blusher, conferring a glossy make-up.

### EXAMPLE 9

A lipstick having the following composition was prepared:

| | | |
|---|---|---|
| Castor oil | 72 | g |
| Octacosanyl stearate | 8 | g |
| Particles of LC polymer which are sold under the name | 10 | g |
| “Helicone ® HC Maple” by Wacker | | |

After application to the lips, a very glossy make-up was obtained.

What is claimed is:

1. A cosmetic composition comprising, in a cosmetically acceptable medium, particles of a liquid crystal polymer, wherein the liquid crystal polymer can be obtained by polymerization of a mixture of monomers comprising:
a) at least one first monomer A of formula (I)
Y¹-A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₀-C₂₀ alkylthio, (C₁-C₂₀)alkylcarbonyl, (C₀-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms.

2. The composition according to claim 1, wherein Z is a chiral divalent group comprising from 4 to 20 carbon atoms.

3. The composition according to claim 2, wherein Z is a chiral divalent group comprising from 4 to 10 carbon atoms.

4. The composition according to claim 1, wherein Z is a chiral divalent group resulting from a group chosen from dianhydrohexites, hexoses, pentoses, binaphthyl derivatives, biphenyl derivatives, tartaric acid derivatives and glycols, which are optically active.

5. The composition according to claim 1, wherein the liquid crystal polymer can be obtained by polymerization:
a) of at least one first monomer A of formula (I) Y¹A¹-M¹-A²-Y²
in which
i) Y¹ and Y², which may be identical or different, are each a group chosen from acrylate and methacrylate groups,
ii) A¹ and A², which may be identical or different, are each a group of formula —CₙH₂ₙ—, in which n is an integer ranging from 1 to 20;
iii) M¹ is a group of general formula (I′)
—R¹—X¹—R²—X²—R³—X³—R⁴—, in which R¹ and R⁴ are each —O—, R² and R³ are each a —COO— group and X¹, X² and X³ are each a 1,4-phenylene group, wherein the carbonyl group —CO— of R² and of R³ are bonded to the X¹ group and to the X³ group respectively, and
b) of at least one chiral second monomer B of formula (II) V¹-W¹-Z-W²-V², in which
i) V¹ is a group chosen from acrylate and methacrylate groups, and V² is a group chosen from C₁-C₂₀ alkyl groups, C₁-C₂₀ alkoxy groups, (C₁-C₂₀)alkoxycarbonyl and —OH groups,
ii) W¹ is a divalent group of formula —X′¹—CO—O—,
W² is a divalent group of formula —O—CO—X′¹—,
in which X′¹ is a 1,4-phenylene group,
and Z is a chiral group comprising two bonds, resulting from the dianhydrohexite groups.

6. The composition according to claim 5, wherein in defining the group V², the C₁-C₂₀ alkoxy groups are a methoxy group.

7. The composition according to claim 5, wherein Y¹ and Y² are each a group chosen from acrylate groups.

8. The composition according to claim 5, wherein A¹ and A², which may be identical or different, are each a group of formula —CₙH₂ₙ—, in which n is an integer ranging from 2 to 6.

9. The composition according to claim 8, wherein A¹ and A², which may be identical or different, are each a group of formula —CₙH₂ₙ— in which n is an integer equal to 4.

10. The composition according to claim 5, wherein V¹ is a group chosen from acrylate groups and V² is a group chosen from C₁-C₄ alkoxy groups.

11. The composition according to claim 10, wherein the C₁-C₄ alkoxy groups are a methoxy group.

12. The composition according to claim 5, wherein Z is a divalent radical of formula:

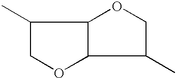

13. The composition according to claim 1, wherein the mixture of monomers comprises the first monomer A in a concentration ranging from about 70 to about 99% by weight and the second monomer B in a concentration ranging from about 1 to about 30% by weight relative to the total weight of the first monomer A and the second monomer B.

14. The composition according to claim 13, wherein the mixture of monomers comprises the first monomer A in a concentration ranging from about 90 to about 95% by weight and the second monomer B in a concentration ranging from about 5 to about 10% by weight relative to the total weight of the first monomer A and the second monomer B.

15. The composition according to claim 1, wherein the liquid crystal polymer exhibits a helical pitch of greater than 450 nm.

16. The composition according to claim 15, wherein the liquid crystal polymer exhibits a helical pitch of ranging from 455 nm to 5 000 nm.

17. The composition according to claim 16, wherein the liquid crystal polymer exhibits a helical pitch of ranging from 455 nm to 1 000 nm.

18. The composition according to claim 17, wherein the liquid crystal polymer exhibits a helical pitch of ranging from 455 nm to 650 nm.

19. The composition according to claim 1, wherein the mixture of the first monomer A and the second monomer B comprises polymerizable groups, at least 90% of which are present in monomers comprising at least two polymerizable groups, in a concentration ranging from about 3.2 to about 15 mmol/g.

20. The composition according to claim 1, wherein the first monomer A has a weight-average molecular weight ranging from about 150 to about 800.

21. The composition according to claim 20, wherein the first monomer A has a weight-average molecular weight ranging from about 460 to about 625.

22. The composition according to claim 1, wherein the second monomer B has a weight-average molecular weight ranging from about 500 to about 1 000.

23. The composition according to claim 22, wherein the second monomer B has a weight-average molecular weight ranging from about 500 to about 700.

24. The composition according to claim 1, wherein the liquid crystal polymer has a weight-average molecular weight of less than 625.

25. The composition according to claim 1, wherein the particles of a liquid crystal polymer have the largest dimension ranging from about 1 μm to about 3 mm.

26. The composition according to claim 25, wherein the particles of a liquid crystal polymer have the largest dimension ranging from about 30 μm to about 500 μm.

27. The composition according to claim 1, wherein the particles of a liquid crystal polymer are in the form of platelets.

28. The composition according to claim 1, wherein the particles of a liquid crystal polymer are present in a concentration ranging from about 0.1% to about 99% by weight relative to the total weight of the composition.

29. The composition according to claim 28, wherein the particles of a liquid crystal polymer are present in a concentration ranging from about 0.1% to about 60% by weight relative to the total weight of the composition.

30. The composition according to claim 29, wherein the particles of a liquid crystal polymer are present in a concentration ranging from about 1% to about 30% by weight relative to the total weight of the composition.

31. The composition according to claim 30, wherein the particles of a liquid crystal polymer are present in a concentration ranging from about 5% to about 15% by weight relative to the total weight of the composition.

32. The composition according to claim 1, further comprising a cosmetic medium chosen from hydrophilic cosmetic mediums and lipophilic cosmetic mediums.

33. The composition according to claim 1, further comprising water or a mixture of water and of at least one hydrophilic organic solvent.

34. The composition according to claim 1, further comprising a fatty phase.

35. The composition according to claim 34, wherein the fatty phase comprises at least one ingredient chosen from oils, waxes, pasty fatty substances, and gums.

36. The composition according to claim 1, further comprising an organic solvent.

37. The composition according to claim 1, further comprising at least one film-forming polymer.

38. The composition according to claim 37, wherein the at least one film-forming polymer is chosen from vinyl polymers, polyurethanes, polyesters, polyamides, polyureas and cellulose polymers.

39. The composition according to claim 37, wherein the at least one film-forming polymer is present in a concentration on a dry basis of the polymer ranging from about 0.1% to about 60% by weight relative to the total weight of the composition.

40. The composition according to claim 39, wherein the at least one film-forming polymer is present in a concentration on a dry basis of the polymer ranging from about 0.5% to about 40% by weight relative to the total weight of the composition.

41. The composition according to claim 40, wherein the at least one film-forming polymer is present in a concentration on a dry basis of the polymer ranging from about 1% to about 30% by weight relative to the total weight of the composition.

42. The composition according to claim 1, further comprising at least one additional colouring material different from the particles of a liquid crystal polymer.

43. The composition according to claim 42, wherein the at least one additional colouring material is chosen from pigments, pearlescence agents and water-soluble and fat-soluble dyes.

44. The composition according to claim 42, wherein the at least one additional colouring material is a pigment chosen from titanium dioxides, zirconium oxides, cerium oxides, zinc oxides, iron oxides, chromium oxides, manganese violet, ultramarine blue, chromium hydrate, ferric blue, aluminium powder, copper powder, carbon black, pigments of D & C type, and lakes based on cochineal carmine and on barium, strontium, calcium and aluminium.

45. The composition according to claim 43, wherein the at least one additional colouring material is chosen from pigments present in a concentration ranging from about 0.01% to about 15% by weight relative to the total weight of the composition.

46. The composition according to claim 45, wherein the at least one additional colouring material is chosen from pigments present in a concentration ranging from about 0.01% to about 10% by weight relative to the total weight of the composition.

47. The composition according to claim 46, wherein the at least one additional colouring material is chosen from pigments present in a concentration ranging from about 0.02% to about 5% by weight relative to the total weight of the composition.

48. The composition according to claim 42, wherein the at least one additional colouring material is a pearlescence agent chosen from mica covered with titanium oxide, mica covered with bismuth oxychloride, titanium oxide-coated mica covered with iron oxides, titanium oxide-coated mica covered with ferric blue, titanium oxide-coated mica covered with chromium oxide, titanium oxide-coated mica covered with an organic pigment, and pearlescent pigments based on bismuth oxychloride.

49. The composition according to claim 48, wherein the at least one additional colouring material is chosen from pearlescence agents present in a concentration ranging from about 0.01% to about 25% by weight relative to the total weight of the composition.

50. The composition according to claim 49, wherein the at least one additional colouring material is chosen from pearlescence agents present in a concentration ranging from about 0.01% to about 10% by weight relative to the total weight of the composition.

51. The composition according to claim 50, wherein the at least one additional colouring material is chosen from pearlescence agents present in a concentration ranging from about 0.02% to about 5% by weight relative to the total weight of the composition.

52. The composition according to claim 1, further comprising at least one cosmetic ingredient chosen from fillers, vitamins, thickeners, surfactants, trace elements, moisturizers, softeners, sequestering agents, fragrances, basifying or acidifying agents, preservatives, antioxidants, and UV screening agents.

53. The composition according to claim 1, wherein the composition is provided in a form chosen from forms of a nail varnish, mascara, eyeliner, product for making up a lip, foundation, blusher, eye shadow, product for an eyebrow and product for making up a body.

54. The composition according to claim 53, wherein the composition is provided in the form of a nail varnish.

55. A cosmetic process for making up a keratinous substance, comprising applying to the keratinous substance a composition comprising, in a cosmetically acceptable medium, particles of a liquid crystal polymer, wherein the liquid crystal polymer can be obtained by polymerization of a mixture of monomers comprising:
a) at least one first monomer A of formula (I)
Y¹-A-M¹-A 2-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms.

56. A cosmetic process for making up a keratinous substance comprising applying to the keratinous substance a first coat of a first composition comprising, in a cosmetically acceptable medium, at least one colouring material and then applying to at least one portion of the said first coat, a second coat of a second composition comprising, in a cosmetically acceptable medium, particles of a liquid crystal polymer, wherein the liquid crystal polymer can be obtained by polymerization of a mixture of monomers comprising:
a) at least one first monomer A of formula (I)
Y¹A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V1-A′1-W1-Z-W2-A′2-V2, in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms,
wherein the first composition does not comprise particles of a liquid crystal polymer like those present in the second composition.

57. The process according to claim 56, wherein in the first composition, the at least one colouring material is chosen from pigments, pearlescence agents, and water-soluble and fat-soluble dyes.

58. The process according to claim 56, wherein the first composition further comprises at least one film-forming polymer.

59. The process according to claim 56, wherein the first composition further comprises at least one cosmetic ingredient chosen from fillers, vitamins, thickening agents, surfactants, trace elements, moisturizers, softeners, sequestering agents, fragrances, basifying or acidifying agents, preservatives, antioxidants, and UV screening agents.

60. The process according to claim 56, wherein the first composition is provided in a form chosen from forms of a nail varnish, mascara, eyeliner, product for making up a lip, foundation, blusher, eye shadow, product for an eyebrow and product for making up a body.

61. The process according to claim 60, wherein the first composition is provided in the form of a nail varnish.

62. A make-up kit comprising:
a first composition comprising, in a cosmetically acceptable medium, at least one colouring material, and
a second composition comprising, in a cosmetically acceptable medium, particles of a liquid crystal polymer, wherein the liquid crystal polymer can be obtained by polymerization of a mixture of monomers comprising:
a) at least one first monomer A of formula (I)
Y¹-A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms,
wherein the first composition does not comprise particles of liquid crystal polymer like those present in the second composition, and
the first and second compositions are packaged in separate containers.

63. The make-up kit according to claim 62, wherein in the first composition, the at least one colouring material is chosen from pigments, pearlescence agents, and water-soluble and fat-soluble dyes.

64. The make-up kit according to claim 62, wherein the first composition further comprises at least one film-forming polymer.

65. The make-up kit according to claim 62, wherein the first composition further comprises at least one cosmetic ingredient chosen from fillers, vitamins, thickeners, surfactants, trace elements, moisturizers, softeners, sequestering agents, fragrances, basifying or acidifying agents, preservatives, antioxidants, and UV screening agents.

66. The make-up kit according to claim 62, wherein the first composition is provided in a form chosen from forms of a nail varnish, mascara, eyeliner, product for making up a lip, foundation, blusher, eye shadow, product for an eyebrow and product for making up a body.

67. The make-up kit according to any one of claim 66, where in the first composition is provided in the form of a nail varnish.

68. A make-up support comprising a make-up positioned on a support, said make-up comprising particles of a liquid crystal polymer, wherein the liquid crystal polymer can be obtained by polymerization of a mixture of monomers comprising:
a) at least one first monomer A of formula (I)
Y¹-A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms,
and said support is chosen from false nails, false eyelashes, toupees, wigs, and adherent discs and patches for skin or lip.

69. A process for manufacturing a glossy make-up composition comprising including in the composition particles of a liquid crystal polymer, wherein the liquid crystal polymer can be obtained by polymerization of a mixture of monomers comprising:
a) at least one first monomer A of formula (I)
Y¹A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms.

70. The composition according to claim 1, wherein the cycloalkylene groups are 1,4-cyclohexylene groups.

71. The composition according to claim 1, wherein the arylene groups are 1,4-phenylene groups.

72. A cosmetic composition comprising, in a cosmetically acceptable medium, particles of a liquid crystal polymer, wherein the liquid crystal polymer comprises
a) at least one residue of a first monomer A of formula (I)
Y¹-A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one residue of a chiral second monomer B of formula (II) V¹-A′¹-W¹-Z-W²-A′²-V², in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms.

73. A make-up support comprising a make-up positioned on a support, wherein said make-up comprises a first coat on the support of a first composition comprising, in a cosmetically acceptable medium, at least one colouring material and a second coat on at least one portion of the first coat of a second composition comprising, in a cosmetically acceptable medium, particles of a liquid crystal polymer, wherein the liquid crystal polymer can be obtained by polymerization of a mixture of monomers comprising:
a) at least one first monomer A of formula (I)
Y¹-A¹-M¹-A²-Y²
in which
i) Y¹ and Y², which may be identical or different, are each a polymerizable group chosen from acrylate and methacrylate groups, and epoxy, isocyanate, hydroxyl, vinyl ether (—O—CH═CH₂) and vinyl ester (—CO—O—CH═CH₂) groups,
ii) A¹ and A², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) M¹ is a group of general formula (I′)
—R¹—X¹—R²—X²—R³—X³—R⁴—, wherein R¹, R², R³ and R⁴, which may be identical or different, are each a divalent group chosen from —O—, —COO—, —CONH—, —CO—, —S—, —C≡C—, —CH═CH—, —N═N— and —N═N(O)— groups, and it being possible for —R²—X²—R³— or —R²—X²— or —R²—X²—R³—X³— also to be a single covalent bond, and X¹, X² and X³, which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms, and
b) at least one chiral second monomer B of formula (II) V1-A′1-W1-Z-W2-A′2-V2, in which
i) V¹ and V², which may be identical or different, are each a group chosen from acrylate and methacrylate groups, epoxy groups, vinyl ether and vinyl ester groups, isocyanate groups, C₁-C₂₀ alkyl, C₁-C₂₀ alkoxy, C₁-C₂₀ alkylthio, (C₁-C₂₀)alkoxycarbonyl, (C₁-C₂₀)alkylthiocarbonyl, —OH, —F, —Cl, —Br, —I, —CN, —NO₂, formyl, acetyl groups, and wherein said C₁-C₂₀ alkyl, C₁-C₂₀ alkoxy, C₁-C₂₀ alkylthio groups include at least one entity chosen from oxygen and sulphur atoms and an ester group (—CO—O—),
and at least one of V¹ and V² is a polymerizable group chosen from acrylate and methacrylate groups, epoxy groups, and isocyanate, hydroxyl, vinyl ether (—O—CH═CH₂) and vinyl ester (—CO—O—CH═CH₂) groups,
ii) A′¹ and A′², which may be identical or different, are each a group of formula —CₙH₂ₙ—, wherein n is an integer ranging from 0 to 20, and it being possible for at least one methylene group of the said —CₙH₂ₙ— groups to be replaced by at least one oxygen atom, and
iii) W¹ and W², which may be identical or different, are each a divalent group chosen from groups of general formula —R′¹—X′¹—R′²—X′²—R′³—, wherein R′¹, R′² and R′³, which may be identical or different, are each a divalent group chosen from —O—, —COO—, —CONH—, —CO—, —S—, —C≡C—, —CH═CH—, —N═N— and —N═N(O)— groups, and R′¹, R′², R′³ or R′²—X′² can also be a single covalent bond, and X′¹ and X′², which may be identical or different, are each a group chosen from cycloalkylene groups comprising from 3 to 10 carbon atoms, which can be optionally substituted by at least one group chosen from —B¹, —B² and —B³ groups, wherein —B¹, —B² and —B³, which may be identical or different, are each chosen from C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups and wherein said C₁-C₂₀ alkyl, C₁-C₂₀ alkoxy, C₁-C₂₀ alkylthio may include at least one entity chosen from oxygen and sulphur atoms and ester groups, and arylene groups comprising from 6 to 10 atoms, which are optionally substituted by at least one group chosen from said —B¹, —B² and —B³ groups and heteroarylene groups comprising an aryl nucleus comprising from 6 to 10 atoms which are optionally substituted by at least one group chosen from said —B¹, —B² and —B³ groups, the said heteroarylene groups comprising from 1 to 3 heteroatoms chosen from O, N and S atoms,
and Z is a chiral divalent group comprising at least 4 carbon atoms,
wherein the first composition does not comprise particles of a liquid crystal polymer like those present in the second composition,
and said support is chosen from false nails, false eyelashes, toupees, wigs, and adherent discs and patches for skin or lip.
